(19) United States
(12) Patent Application Publication
(10) Pub. No.: US 2011/0144578 A1
(43) Pub. Date: Jun. 16, 2011

(54) HYDROPHOBIC THERAPUEUTIC AGENT AND SOLID EMULSIFIER COATING FOR DRUG COATED BALLOON

(76) Inventors: Stephen Pacetti, San Jose, CA (US); John Stankus, Campbell, CA (US)

(21) Appl. No.: 12/636,246

(22) Filed: Dec. 11, 2009

(51) Int. Cl. A61M 29/02; B05D 3/02

(52) U.S. Cl. 604/96.01; 427/2.3

(57) ABSTRACT

The disclosed subject matter is directed to a coated medical device such as a balloon or stent and methods of manufacturing the device, where the device has a working length disposed between a distal end and a proximal end thereof; and a coating applied to at least a length of the body. The coating includes a hydrophobic therapeutic agent having a water solubility less than about 15.0 μg/ml and an emulsifier that is a solid at ambient temperature.

### FIELD OF THE INVENTION

The disclosed subject matter is related to the delivery of insoluble drugs from an insertable medical device. More particularly, the disclosed subject matter relates to a medical device including a balloon having a coating of a therapeutic agent with low water solubility and an emulsifier at a low drug to emulsifier ratio.

### BACKGROUND OF THE INVENTION

Atherosclerosis is a syndrome affecting arterial blood vessels. It is a chronic inflammatory response in the walls of arteries, which is in large part due to the accumulation of lipid, macrophages, foam cells and the formation of plaque in the arterial wall. Atherosclerosis is commonly referred to as hardening of the arteries although the pathophysiology of the disease manifests itself with several different types of lesions ranging from fibrotic to lipid laden to calcific. Angioplasty is a vascular interventional technique involving mechanically widening an obstructed blood vessel, typically caused by atherosclerosis.

During angioplasty, a catheter having a tightly folded balloon is inserted into the vasculature of the patient and is passed to the narrowed location of the blood vessel at which point the balloon is inflated to a desired size and pressure using an inflation medium, usually a radiopaque contrast media. Percutaneous coronary intervention (PCI), commonly known as coronary angioplasty, is a therapeutic procedure to treat the stenotic coronary arteries of the heart, often a hallmark of coronary arterial disease. In contrast, peripheral angioplasty, commonly known as percutaneous transluminal angioplasty (PTA), refers to the use of mechanical widening of blood vessels other than the coronary arteries. PTA is most commonly used to treat narrowing of the arteries of the leg, especially, the iliac, external iliac, superficial femoral and popliteal arteries. PTA can also treat narrowing of veins and other blood vessels.

Although the blood vessel is often successfully widened by angioplasty, sometimes the treated wall of the blood vessel undergoes vasospasm, or abrupt closure after balloon inflation or dilatation, causing the blood vessel to collapse after the balloon is deflated or shortly thereafter. One solution to such collapse is stenting the blood vessel to prevent collapse. A stent is a device, typically a metal tube or scaffold, that is inserted into the blood vessel after, or concurrently with angioplasty, to hold the blood vessel open.

While the advent of stents eliminated many of the complications of abrupt vessel closure after angioplasty procedures, within about six months of stenting, a re-narrowing of the blood vessel can form, which is a condition known as restenosis. Restenosis was discovered to be a response to the injury of the angioplasty procedure and is characterized by a growth of smooth muscle cells—analogous to a scar foaming over an injury. As a solution, drug eluting stents were developed to address the reoccurrence of the narrowing of blood vessels. One example of a drug eluting stent is a metal stent that has been coated with a drug that is known to interfere with the process of restenosis. A potential drawback of certain drug eluting stents is known as late stent thrombosis, which is an event in which blood clots inside the stent.

Drug delivery balloons are believed to be a viable alternative to drug eluting stents in the treatment of atherosclerosis. In a study which evaluated restenosis, and the rate of major adverse cardiac events such as heart attack, bypass, repeat stenosis, or death in patients treated with drug eluting balloons and drug eluting stents, the patients treated with drug eluting balloons experienced only 3.7 percent restenosis and 4.8 percent MACE (major adverse coronary events) as compared to patients treated with a drug eluting stent, in which restenosis was 20.8 percent and 22.0 percent MACE rate. (See, PEPCAD II study, Rotenburg, Germany).

Although drug eluting balloons are a viable alternative, and in some cases can have greater efficacy than drug eluting stents as suggested by the PEPCAD II study, drug delivery balloons present unique challenges. In particular, the drug needs to be released from the balloon surface, or the coating needs to be transferred to the blood vessel wall when the balloon is expanded inside the blood vessel. For coronary procedures, the balloon is typically inflated for less than one minute, typically about thirty seconds. The balloon can be able to be expanded for a longer period of time for peripheral procedure, however typically even for peripheral procedures the balloon is expanded for less than 5 minutes. Due to the very short duration of contact of the drug coated balloon surface with the blood vessel wall, the balloon coating must exhibit therapeutic agent transfer efficiency and/or efficient drug release during inflation, which is within minutes.

Further, the amount of drug that is released into the systemic circulation needs to be minimized. Thus, there are challenges specific to drug delivery via a drug coated (or drug eluting) balloon because of the necessity of a short inflation time, and therefore time for drug or coating transfer--a challenge not presented by a drug eluting stent, which remains in the patient's vasculature once implanted.

### SUMMARY OF INVENTION

The disclosed subject matter includes a drug delivery balloon which exhibits improved coating transfer efficiency to the wall of a blood vessel and/or increased uptake of highly water insoluble therapeutic agent into a blood vessel wall. Generally, the balloon disclosed herein has a coating applied to at least a length of the balloon surface. The coating includes a therapeutic agent with low water solubility, for example, a therapeutic agent having a solubility in an aqueous solution, such as phosphate buffered saline, of less than about 15 μg/ml, and an emulsifier. The emulsifier has properties of a solid at ambient temperature. In one embodiment, the ratio of therapeutic agent to emulsifier is 3:1, or less than 3:1.

It has been determined that a drug delivery balloon having such a coating exhibits certain improvements including: coating integrity enhanced uptake of therapeutic agent and transfer efficiency to the vessel wall. For a balloon coating, coating integrity refers to the coating remaining on the balloon during the necessary operations to which a drug delivery balloon is subjected. Such methods include the folding, pressing and sheathing of a coated balloon. In these methods, the balloon coating is dry, and it is desired for the coating to stay on the balloon. Next, when in use by a physician, the drug delivery balloon is unsheathed, passed through a hemostatic valve, passed through and introducer or guide sheath, and then tracked through the vasculature to the desired treatment site. In all of these methods, most of which are in-vivo, good coating integrity translates into the coating staying on the balloon so that a significant fraction is still present when the balloon reaches the lesion.

Further, the therapeutic agent in the coating has improved solubility when placed in-vivo. By this, it is meant that the therapeutic agent solubilizes in-vivo within the time frame of balloon inflation, typically from about 30 to about 60 seconds or less and improved drug delivery to the vessel tissue region of interest occurs.

The therapeutic agent of the disclosed subject matter is hydrophobic and has a relatively low water solubility of less than about 15 μg/ml in solution. In one embodiment, the therapeutic agent is a cytostatic drug. For example, and not by way of limitation, the therapeutic agent includes zotarolimus, everolimus, sirolimus, biolimus, novolimus, myolimus, temsirolimus, deforolimus, paclitaxel, docetaxel, or protaxel, or any combination thereof.

The emulsifier has properties of a solid at ambient temperature. For example, and not limitation, the emulsifier includes Tween 60, Vitamin E, Pluronic F68, Pluronic F127, Poloxamer 407, glycerol monostearate, Ascorbyl palmitate lecithin, egg yolk, phospholipid, phosphatidylcholine, polyethylene glycol-phosphatidyl ethanolamine conjugate or a combination thereof. Other examples of emulsifiers include PEG-phospholipid conjugates, such as 1,2-diacyl-sn-glycero-3-phosphoethanolamine-N-[methoxy(polyethylene glycol)-350] (mPEG 350 PE) 18:0 distearoyl, ammonium salt; 1,2-Ddiacyl-sn-glycero-3-phosphoethanolamine-N-[methoxy(polyethylene glycol)-1000] (mPEG 1000 PE) 18:0 distearoyl, ammonium salt; 1,2-diacyl-sn-glycero-3-phosphoethanolamine-N-[methoxy(polyethylene glycol)-2000] (mPEG 2000 PE) 18:0 distearoyl, ammonium salt; 1,2-diacyl-sn-glycero-3-phosphoethanolamine-N-[methoxy(polyethylene glycol)-2000] (mPEG 2000 PE) 16:0 dipalmitoyl, ammonium salt; 1,2-diacyl-sn-glycero-3-phosphoethanolamine-N-[methoxy(polyethylene glycol)-3000](mPEG 3000 PE) 18:0 distearoyl, ammonium salt; and 1,2-diacyl-sn-Glycero-3-phosphoethanolamine-N-[methoxy(polyethylene glycol)-2000] (mPEG 2000 PE) 18:0 distearoyl, sodium salt.

In accordance with a further aspect of the disclosed subject matter, the coating includes a plasticizer. For example, the plasticizer has properties of a liquid at ambient temperature. Some non-limiting examples of the plasticizer include polysorbate, such as Tween 20 or Tween 80, an oil, such as but not limited to soybean oil, peanut oil, safflower oil, poppyseed oil, vegetable oil, cottonseed oil, castor oil, and almond oil. Other plasticizers include benzyl alcohol, DMSO, NMP, glycerol, propylene glycol, Cremophor EL, Vitamin E, tocopherol, ethyl lactate, or liquid PEG. The plasticizer preferably is no more than about 20% by weight of the solids of the coating.

Preferably, the coated balloon is disposed on a catheter body for insertion of the drug delivery balloon to the vasculature of a patient. The catheter can include an elongated tubular member having a proximal end, a distal end and a lumen there between. In one embodiment, the catheter has an over-the-wire configuration. In another embodiment, catheter has a rapid exchange configuration.

In accordance with another aspect of the disclosed subject matter, the coated balloon includes a stent disposed thereon. In one embodiment, the coating has a thickness of about 0.5 to about 20 μm.

In another aspect of the disclosed subject matter, a method of manufacturing a drug delivery balloon is provided. In this regard, a catheter including an expandable balloon member is provided and a solution comprising a hydrophobic therapeutic agent having a water solubility less than about 15.0 ug/ml and an emulsifier having properties of a solid at ambient temperature is applied to the balloon member. In one embodiment, the solution is applied to the balloon in an expanded state. In this regard, the balloon is expanded under low pressures of about 0.2 atm to about 9 atm. Alternatively, the balloon can be coated with the solution in its deflated state. The balloon can be folded or unfolded during the application of the therapeutic agent solution to the balloon to define the coated balloon. The balloon member can be rotated and/or translated during application of the therapeutic agent solution to form a coating having desired thickness on the surface of the balloon member. Otherwise, the balloon is held stationary, or only rotated, and the coating applicator moved relative to the balloon.

The balloon can then be heated to remove remaining solvent from the coating. The heating can include baking the balloon for example at a temperature of about 30° C. to about 110° C., more preferable from about 40° C. to about 80° C., and for example, greater than or about 50° C. Generally, the balloon is heated for about 15 to about 60 minutes or sufficient time to evaporate remaining solvent from the coating. The balloon can be baked in a forced air convection oven, gravity convection oven, or vacuum oven. It can be dried by placing it in a stream of heated air, nitrogen, argon, or other inert gas. Other techniques include drying by infrared radiation heating, microwave drying, or drying in a fluidized bed. If desired, a plasticizer can be added to the solution to render the coating less brittle.

In one embodiment, the method includes applying a coating to at least a length of an expandable member to define a thickness of about 0.5 to about 20 μm, and preferably from about 2 to about 10 μm, and disposing the expandable member on a catheter. The method can further include preparing a pre-coating mixture for example by mixing a therapeutic agent and an emulsifier, and conditioning the pre-coating to form a porous coating by a phase inversion technique. Additionally, or alternatively, the method can include creating a coating to which a porogen is added to define a porous coating for application to the balloon.

For example, the porous coating can be created by phase inversion techniques. In another embodiment, the porous coating is created by introduction of a porogen to a mixture including a therapeutic agent to be applied to the delivery device. In another embodiment, the porogen is removed from the coating prior to application of the coating to the delivery device.

It is to be understood that both the foregoing description is exemplary and is intended to provide further explanation of the disclosed subject matter claimed to a person of ordinary skill in the art. The accompanying drawings are included to illustrate various embodiments of the disclosed subject matter to provide a further understanding of the disclosed subject matter. The exemplified embodiments of the disclosed subject matter are not intended to limit the scope of the claims.

### BRIEF DESCRIPTION OF DRAWINGS

FIG. 1 depicts a representative embodiment of a medical device of the disclosed subject matter, which is shown as a catheter with a balloon for illustration and not limitation.

FIG. 2 is a graph illustrating the results from a comparative study of drug delivery balloons and percentage of drug remaining on the balloon in a porcine coronary and mammary artery pharmacokinetic model, wherein the amount of zotarolimus (μg) on the balloons (y-axis) is measured against the percent of balloon dose (x-axis).

FIG. 3 is a graph illustrating therapeutic agent and percent initial balloon dose remaining in tissue after delivery in a porcine coronary and mammary pharmacokinetic model using an embodiment of the disclosed subject matter.

FIG. 4 is a graph illustrating the results from a comparative study of drug delivery balloons and percentage of drug remaining on the balloon in a porcine iliofemoral artery pharmacokinetic model.

FIG. 5 is an optical micrograph at 50× magnification of a glass slide coating of Zotarolimus/Tween 20 at a 1/2 weight ratio.

FIG. 6 is an optical micrograph at 50× magnification of a glass slide coating of Zotarolimus/PEG-PE at a 1/2 weight ratio.

FIG. 7 is an optical micrograph at 50× magnification of a glass slide coating of Zotarolimus/Vitamin E TGPS at a 2/1 weight ratio.

### DETAILED DESCRIPTION OF EMBODIMENTS

Reference will now be made in detail to the present embodiments of the disclosed subject matter, certain examples of which are illustrated in the accompanying figures. The disclosed subject matter will be described in conjunction with the detailed description of the device. Specific embodiments as described herein are provided as exemplary of the disclosed subject matter, and not by way of limitation.

It is to be noted that the term “a” entity or “an” entity refers to one or more of that entity. For example, a protein refers to one or more proteins or at least one protein. As such, the terms “a”, “an”, “one or more”, and “at least one” can be used interchangeably herein. The terms “comprising,” “ including,” and “having” can also be used interchangeably. In addition, the terms “amount” and “level” are also interchangeable and can be used to describe a concentration or a specific quantity. Furthermore, the term “selected from the group consisting of” refers to one or more members of the group in the list that follows, including mixtures (i.e. combinations) of two or more members.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to +/−20%, preferably up to +/−10%, more preferably up to +/−5%, and more preferably still up to +/−1% of a given value. Alternatively, particularly with respect to biological systems or processes, the term can mean within an order of magnitude, preferably within 5-fold, and more preferably within 2-fold, of a value. With reference to pharmaceutical compositions, the term “about” refers to a range that is acceptable for quality control standards of a product approved by regulatory authorities.

The device and method of the disclosed subject matter can be used for delivery within and/or treating of the lumen of a patient. In particular, the disclosed subject matter is particularly suited for treatment of the cardiovascular system of a patient, such as performance of angioplasty and/or delivery of a therapeutic agent in a relatively short amount of time, such as under about 60 seconds by way of a drug delivery medical balloon. Longer applications can be used as needed or if desired.

Without limitation to the disclosed subject matter, one theory of drug delivery to a vessel wall suggests that there is a dissolution mechanism by which therapeutic agent dissolves from a coated balloon, and the therapeutic agent molecules diffuse to the vessel wall and then permeate into the vessel wall. Using the Stokes-Einstein equation as a simple model, diffusion coefficients are approximately proportional to the molecular weight of a species raised to the −⅓ power. Thus, individual molecules or micelles, which are low in molecular weight, tend to diffuse much faster than microspheres or nanoparticles, which are much higher in molecular weight. Thus, it is advantageous to develop a coating formulation that exhibits high solubility once released. There can be difficulty in formulating drug delivery balloons that must have rapid therapeutic agent release (in seconds) with highly water insoluble therapeutic agents, such as zotarolimus, which has a water solubility of only 0.5 μg/ml, and paclitaxel, which has similar low water solubility.

It has been determined that balloon coating formulations having a liquid emulsifier can suffer from certain drawbacks. In this regard, effective solubilization of a therapeutic agent with low water solubility by a liquid emulsifier requires a ratio of emulsifier to drug greater than unity (i.e., greater than 1:1). Thus, it takes several molecules of liquid emulsifier to surround and solubilize a highly water insoluble therapeutic agent. In opposition to this, liquid emulsifiers require a high ratio of drug to emulsifier to avoid being too fluid to stay in place on the surface of the balloon. Adversely, a lower ratio of drug to liquid emulsifier provides a coating exhibiting a tacky, or viscous behavior which is problematic as the coated balloon sticks to processing equipment and/or to its own folds, and/or to the protective sheath.

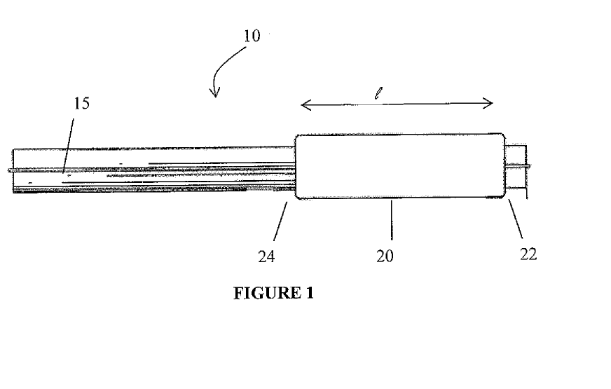

In accordance with one aspect of the disclosed subject matter, a balloon for delivering a highly water insoluble therapeutic agent is provided. The balloon includes a body having a working length disposed between distal and proximal ends of the balloon, such as between first and second cone portions, and a coating applied to at least a length of the balloon. Referring to FIG. 1, for purposes of illustration and not limitation, a device is provided with a drug delivery balloon that exhibits improved coating transfer from the balloon. An exemplary balloon disposed on a catheter is shown in FIG. 1. As depicted in FIG. 1, the device is a catheter 10 including a catheter shaft 15 and balloon 20. The balloon 20 has a distal end 22, a proximal end 24 and a working length “l” therebetween. The catheter includes an elongate shaft having a proximal end, a distal end and at least one lumen therebetween. Preferably, the catheter includes a multilumen shaft such as an inflation lumen and a guidewire lumen. In this regard, multilumen can be arranged in a coaxial or side-by-side configuration. Further, the catheter can be configured as a rapid exchange catheter or an over-the-wire catheter. A wide variety of balloon shapes, sizes, and materials of construction can be used in accordance herewith. Possible materials and construction are described in further detail below.

As used herein the phrase “highly water insoluble” or “low water solubility” means an agent having a solubility in water of less than about 15.0 μg/ml. The term “hydrophobic” relates to the therapeutic agent being highly water insoluble or having low water solubility. The coating disclosed herein includes a therapeutic agent with low water solubility and an emulsifier to dissolve the therapeutic agent. The emulsifier has properties of a solid at ambient temperature. The therapeutic agent and emulsifier form micelles that solubilize the therapeutic agent in aqueous solution.

It has been determined that utilization of a solid emulsifier in the appropriate ratio provides an improved coating, which exhibits improved coating integrity when dry, solubilizes the therapeutic agent in vivo as opposed to exhibiting drug fracture into pieces and/or chunks, enhances therapeutic agent uptake, and transfers efficiency by solubilizing the therapeutic agent so it can diffuse into the vessel wall. In one embodiment, the coating has a thickness between about 0.5 μm to about 20 μm, and more preferably, a thickness of about 2 μm to about 10 μm.

As disclosed herein, the ratio of therapeutic agent to emulsifier is about 3:1, or less than 3:1. The therapeutic agent can be any hydrophobic therapeutic agent. However, preferably, the therapeutic agent is an antiproliferative or a cytostatic drug. The term “cytostatic” as used herein means a drug that mitigates cell proliferation but allows cell migration. The term “antiproliferative” as used herein means a drug used to inhibit cell growth, such as chemotherapeutic drugs. Various therapeutic agents with low water solubility can be used in the coating. For the purpose of illustration and not by way of limitation, the therapeutic agent can include zotarolimus, everolimus, sirolimus, biolimus, novolimus, myolimus, temsirolimus, deforolimus, paclitaxel, docetaxel, protaxel, or a combination thereof.

The emulsifier can be selected from various emulsifiers that have properties of a solid substance under ambient conditions. For example, suitable emulsifiers include Tween 60, Vitamin E, Pluronic F68, Pluronic F127, Poloxamer 407, glycerol monostearate, Ascorbyl palmitate lecithin, egg yolk, phospholipid, phosphatidylcholine, polyethylene glycol-phosphatidyl ethanolamine conjugate or a combination thereof. Other examples of emuslifiers include PEG-phospholipid conjugates, such as 1,2-diacyl-sn-glycero-3-phosphoethanolamine-N-[methoxy(polyethylene glycol)-350] (mPEG 350 PE) 18:0 distearoyl, ammonium salt,;1,2-Ddiacyl-sn-glycero-3-phosphoethanolamine-N-[methoxy(polyethylene glycol)-1000] (mPEG 1000 PE) 18:0 distearoyl, ammonium salt; 1,2-diacyl-sn-glycero-3-phosphoethanolamine-N-[methoxy(polyethylene glycol)-2000] (mPEG 2000 PE) 18:0 distearoyl, ammonium salt; 1,2-diacyl-sn-glycero-3-phosphoethanolamine-N-[methoxy(polyethylene glycol)-2000] (mPEG 2000 PE) 16:0 dipalmitoyl, ammonium salt; 1,2-diacyl-sn-glycero-3-phosphoethanolamine-N-[methoxy(polyethylene glycol)-3000](mPEG 3000 PE) 18:0 distearoyl, ammonium salt; and 1,2-diacyl-sn-Glycero-3-phosphoethanolamine-N-[methoxy(polyethylene glycol)-2000] (mPEG 2000 PE) 18:0 distearoyl, sodium salt.

As disclosed herein, the coating further includes a plasticizer. The plasticizer can be a low molecular weight solvent, oil, or a second liquid emulsifier or surfactant. Without limitation to the disclosed subject matter, plasticizers can include benzyl alcohol, benzyl benzoate, ethanol, DMSO, NMP, glycerol, propylene glycol, Cremophor EL, Vitamin E, Tocopherols, ethyl lactate, soybean oil, peanut oil, liquid PEG, poppyseed oil, safflower oil, vegetable oil, cottonseed oil, castor oil, almond oil, Tween 20 or Tween 80. The plasticizer preferably is no more than about 20% by weight of the solids of the coating.

In accordance with the disclosed subject matter, the coating can be applied to a medical device by processes such as dip-coating, pipette coating, syringe coating, air assisted spraying, electrostatic spraying, piezoelectric spraying, electrospinning, direct fluid application, or other means as known to those skilled in the art. The coating can contain the drug homogeneously dissolved or encapsulated in particles. The coating can be applied over at least a length or the entirety of the balloon or medical device. By way of example, and not limitation, certain coating processes that can be used with the instant disclosed subject matter are described in U.S. Pat. No. 6,669,980 to Hansen; U.S. Pat. No. 7,241,344 to Worsham; and U.S. Publication No. 2004/0234748 to Stenzel, the entire disclosures of which are hereby incorporated by reference in their entirety. In accordance with one embodiment of the disclosed subject matter, the medical device is a balloon, wherein and the coating can be applied to the balloon in either a folded or inflated state. Coating characteristics are affected by process variables. For example, for a dip-coating process, coating quality and thickness can vary as an effect of variables such as number of dips, rate of withdrawal, depth of dips, along with drying time and temperature.

In another aspect of the disclosed subject matter, and described further below, a method is provided to coat a medical balloon. The method includes (i) providing a catheter including an expandable balloon member; (ii) and applying a solution comprising a hydrophobic therapeutic agent having a water solubility less than about 15.0 μg/ml and an emulsifier having properties of a solid at ambient temperature to the expandable balloon member; and (iii) heating the balloon to remove solvent.

Reference will now be made for illustration and not limitation to certain exemplary embodiments in accordance with the disclosed subject matter. In a preferred embodiment, a coating comprising 1.0 gm zotarolimus, 1.0 gm Tween 60, 13.6 gm acetone and 2.4 gm ethanol can be formulated and applied to a balloon. After mixing the ingredients of the coating formulation, the resulting solution can be applied to a balloon made of nylon polymers, such as a 6×40 mm Agiltrac balloon catheter (Abbott Vascular, Santa Clara, Calif.) by a direct dispensing method. The balloon can be inflated to 2 atm pressure and can be passed under a fixed dispense tube while being rotated and translated. A dose density of 300 μg/cm² can be achieved by application of 0.0573 ml of solution. After application of the coating solution, the balloon can be baked at a temperature of 50° C. for about 60 minutes to remove remaining solvent.

In a second embodiment, a coating comprising 2.0 gm everolimus, 1.0 gm Vitamin E TPGS, 5.95 gm acetone, and 1.05 gm ethanol can be formulated and applied to a balloon. After mixing the ingredients of the coating formulation, the resulting solution can be applied to a balloon made of nylon polymers, such as a 6×100 mm Agiltrac balloon catheter (Abbott Vascular, Santa Clara, Calif.) by a direct dispensing method. The balloon can be inflated to 2 atm pressure and can be passed under a fixed dispense tube while being rotated and translated. A dose density of 100 μg/cm² can achieved by application of 0.119 ml of solution. After application of the coating solution, the balloon can baked at a temperature of 50° C. for about 30 minutes to remove remaining solvent.

In a third embodiment, a coating comprising 0.5 gm paclitaxel, 0.25 gm Tween 60, 0.075 gm Cremophor EL, 7.8 gm acetone, and 1.375 gm ethanol can be formulated and applied to a balloon. After mixing the ingredients of the coating formulation, the resulting solution can be applied to a balloon made of nylon polymers, such as a 6×100 mm Agiltrac balloon catheter (Abbott Vascular, Santa Clara, Calif.) by a direct dispensing method. The balloon can be inflated to 2 atm pressure and can be passed under a fixed dispense tube while being rotated and translated. A dose density of 300 μg/cm² can be achieved by application of 0.143 ml of solution. After application of the coating solution, the balloon can be baked at a temperature of 50° C. for about 60 minutes to remove remaining solvent.

In a fourth embodiment, a coating comprising 0.25 gm zotarolimus, 0.25 gm 1,2-distearoyl-sn-glycero-3-phosphoethanolamine-N-[methoxy(polyethyleneglycol)-2000] (ammonium salt) (18:0 PEG2000 PE), 2.25 gm methanol and 2.25 gm acetone is formulated and applied to a balloon. After mixing the ingredients of the coating formulation, the resulting solution is applied to a 6×40 mm Agiltrac balloon catheter (Abbott Vascular, Santa Clara, Calif.) by a direct dispensing method. The balloon is inflated to 2 atm pressure and is passed under a fixed dispense tube while being rotated and translated. A dose density of 300 μg/cm² is achieved by application of 64.5 μl of solution. After application of the coating solution, the balloon is baked at a temperature of 50° C. for about 60 minutes to remove remaining solvent.

In another aspect of the disclosed subject matter, less than 10% of the coating remains on the balloon or medical device post delivery into a lumen of a subject. That is, at least 90% of the coating is delivered from the balloon or medical device to the lumen wall. In another embodiment, less than 30% of the coating remains on the balloon after inflation and deflation in the lumen of a subject. In yet another embodiment, less than 30% of the coating remains on the balloon or expandable medical device post removal of the balloon or medical device from the lumen of the subject. Accordingly, more than about 70% of the coating transfers from the balloon to the subject. Preferably, less than 20% of the coating remains on the balloon or medical device post delivery, inflation and deflation, and/or removal from a lumen of a subject. More preferably, less than 10% of the coating remains on the balloon or medical device post delivery, inflation and deflation, and/or removal from a lumen of a subject.

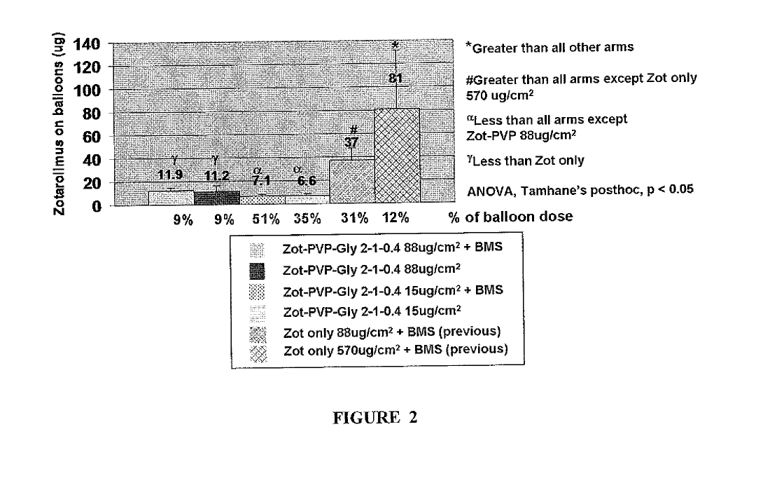

FIG. 2 shows the results from a comparative study in which six different coated balloons were delivered to healthy porcine coronary or mammary arteries in pharmacokinetic models. The coating formulations are tabulated in Table 1.

TABLE 1
Summary of coating formulations studied in the healthy porcine
coronary and mammary artery pharmacokinetic model.

| Bal- | | Dosage of Therapeutic | |
|---|---|---|---|
| loon | Formulation | Agent | |
| 1 | Zotarolimus:PVP:Glycerol (2:1:0.4 | 88 | μg/cm² |
| | weight ratio) | | |
| 2 | Zotarolimus:PVP:Glycerol (2:1:0.4 | 88 | μg/cm² (no stent) |
| | weight ratio) | | |
| 3 | Zotarolimus:PVP:Glycerol (2:1:0.4 | 15 | μg/cm² |
| | weight ratio) | | |
| 4 | Zotarolimus:PVP:Glycerol (2:1:0.4 | 15 | μg/cm² (no stent) |
| | weight ratio) | | |
| 5 | Zotarolimus | 88 | μg/cm² |
| 6 | Zotarolimus | 570 | μg/cm² |

All of the coatings include a therapeutic agent. Most of the coating formulations include excipients or different doses of therapeutic agent to achieve improved efficiency of drug delivery from the balloons. The drug delivery balloons were inserted and inflated for 30 seconds at pressures (based on balloon compliance) needed to inflate the balloons at a 1.2:1 balloon to artery ratio in the coronary and mammary artery porcine animal model. Thereafter, the drug delivery balloons were withdrawn and then the percentage of the initial drug dosage remaining on the balloon surface was calculated. The remaining drug on each of the balloons was assayed by extraction of the balloons in an organic solvent mixture followed by analysis using high performance liquid chromatography (HPLC) and results shown in FIG. 2.

In FIG. 2, groups 1 through 4 (counting bars from left to right) consist of coating formulations each comprising zotarolimus, PVP, and glycerol. In group 1 the dosage of zotarolimus is 88 μg/cm² and the drug:PVP:glycerol is in a ratio of 2:1:0.4. In contrast, group 5 consists of a zotarolimus only formulation at the same dosage of zotarolimus of 88 μg/cm2. When the remaining drug on the balloon is quantified post treatment (inflation), a significantly higher amount of delivered zotarolimus is observed with the zotarolimus:PVP:glycerol formulation compared to zotarolimus alone.

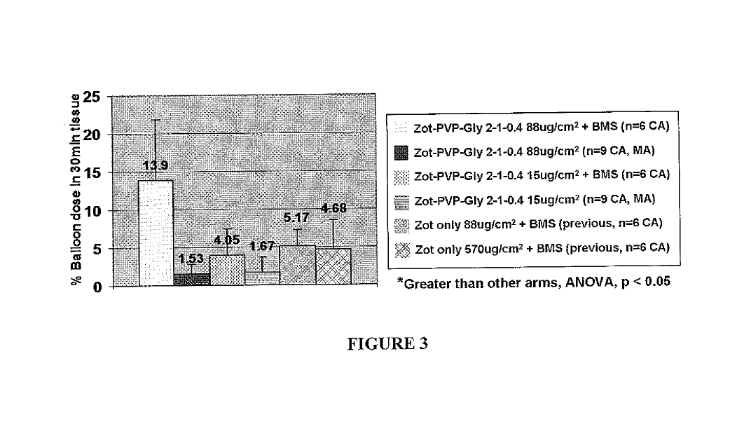

In another aspect of the disclosed subject matter, a drug delivery balloon is provided which exhibits improved tissue uptake of therapeutic agent. FIG. 3 shows the results from a comparative study in which various drug delivery balloons having the formulations of Table 1 were inserted and inflated in porcine coronary and mammary artery pharmacokinetic models. The drug delivery balloons were inserted via femoral access and delivered to either the LCX, LAD, RCA, LIMA or RIMA arteries for a thirty second inflation at pressures (based on balloon compliance) needed to inflate the balloons at a 1.2:1 balloon to artery ratio. The percent of zotarolimus dose per the original balloon dose transferred to the tissue 30 minutes after balloon inflation is depicted in the graph of FIG. 3.

In FIG. 3, group 1 consists of a coating formulations comprising zotarolimus, PVP, and glycerol. In group 1 the dosage of zotarolimus is 88 μg/cm² and the drug:PVP:glycerol is in a ratio of 2:1:0.4. In contrast, group 5 consists of a zotarolimus only formulation at the same dosage of zotarolimus of 88 μg/cm². When the percent of balloon dose transferred to the artery tissue 30 minutes post inflation is quantified, a significantly higher percent transfer of zotarolimus is observed with the zotarolimus:PVP:glycerol formulation compared to zotarolimus alone.

Further, as shown in FIG. 3, it was determined that the tissue uptake has greater improvements when the drug delivery balloon includes a stent crimped on the balloon. In this regard, comparison of groups 1 and 2, each of which have identical coating formulations, exhibited different drug uptake into the tissues of the vessel walls. In particular, group 1 which includes a bare metal stent crimped on the balloon during delivery exhibited greater than six-fold increase in zotarolimus tissue uptake than did group 2, which has no stent disposed on the drug delivery balloon.

Likewise in FIG. 3, group 3 and group 4 each include identical coating formulations, except that group 3 further includes a bare metal stent disposed on the balloon and group 5 has no stent. As shown in FIG. 3, the inclusion of a stent crimped on the balloons in group 3 resulted in a greater than two-fold increase in zotarolimus uptake by the tissue as compared to balloons in group 4. Thus, the inclusion of a bare metal stent disposed on the drug delivery balloon improves tissue uptake of therapeutic agent. Thus, in another aspect of the disclosed subject matter, a drug delivery balloon is provided which exhibits improved tissue uptake of therapeutic agent in one aspect of the disclosed subject matter. The drug delivery balloon comprises a coating applied to at least a length of the balloon surface and a stent disposed on balloon. In this regard, the stent can be a bare metal stent, a coated stent or a drug eluting stent.

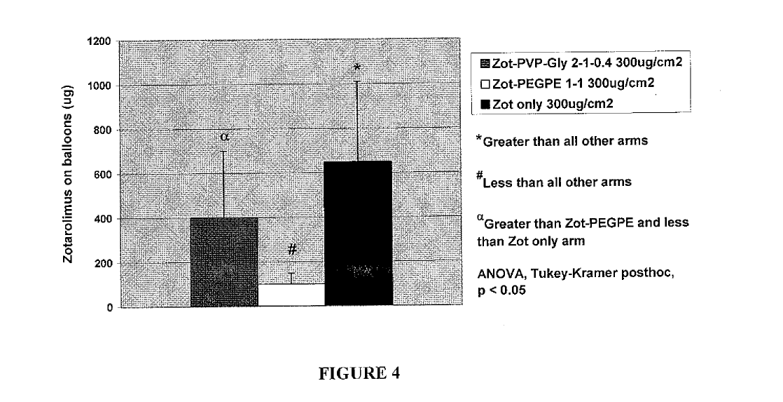

FIG. 4 shows the results from a comparative study in which three different coated balloons were delivered to healthy porcine iliofemoral arteries (iliac, femoral and profunda arteries) in a pharmacokinetic model. The coating formulations are tabulated in Table 2.

TABLE 2
Summary of coating formulations studied in the healthy
porcine iliofemoral artery pharmacokinetic model.

| Bal- | | Dosage of Therapeutic |
|---|---|---|
| loon | Formulation | Agent |
| 1 | Zotarolimus:PVP:Glycerol (2:1:0.4 | 300 μg/cm² (no stent) |
| | weight ratio) | |
| 2 | Zotarolimus:PEGPE (1:1 weight ratio) | 300 μg/cm² (no stent) |
| 3 | Zotarolimus | 300 μg/cm² (no stent) |

All of the coatings include a therapeutic agent. Two out of the three coating formulations include excipients to achieve improved drug delivery from the balloons. The drug delivery balloons were inserted and inflated for 30 seconds in either the iliac, femoral or profunda arteries of the healthy porcine iliofemoral model. Thereafter, the drug delivery balloons were withdrawn and then the percentage of the initial drug dosage remaining on the balloon surface was calculated. The remaining drug on each of the balloons was assayed by extraction of the balloons in an organic solvent mixture followed by analysis using high performance liquid chromatography (HPLC) and results shown in FIG. 4.

In FIG. 4, groups 1 and 2 (counting bars from left to right) consist of coating formulations each comprising excipients designed to better solubilize the hydrophobic therapeutic agent, zotarolimus. Group 1 consists of zotarolimus:PVP:glycerol at a ratio of 2:1:0.4 and dosage of zotarolimus of 300 μg/cm². Group 3 consists of zotarolimus only at a dosage of 300 μg/cm². The amount of zotarolimus remaining on the balloon is significantly less for Group 1 than the amount of zotarolimus remaining on the balloons from Group 3 indicating increased drug delivery from the balloons. Group 2 consists of zotarolimus:PEG-PE at a ration of 1:1. Group 2 is significantly less than either groups 1 or 3 indicating further improved drug delivery from the balloons with the Zot:PEG-PE formulation attributed to the solubilizing effect of the PEG-PE solid emulsifier.

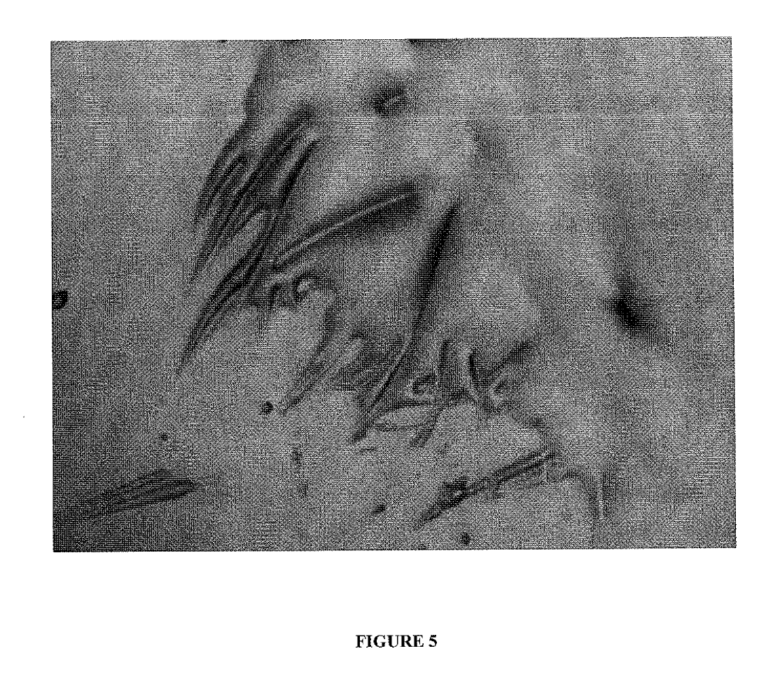

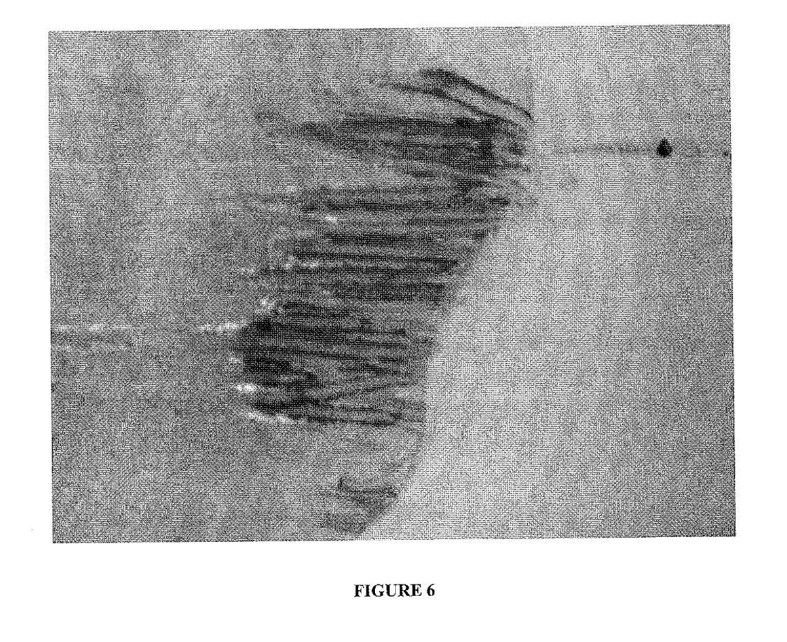

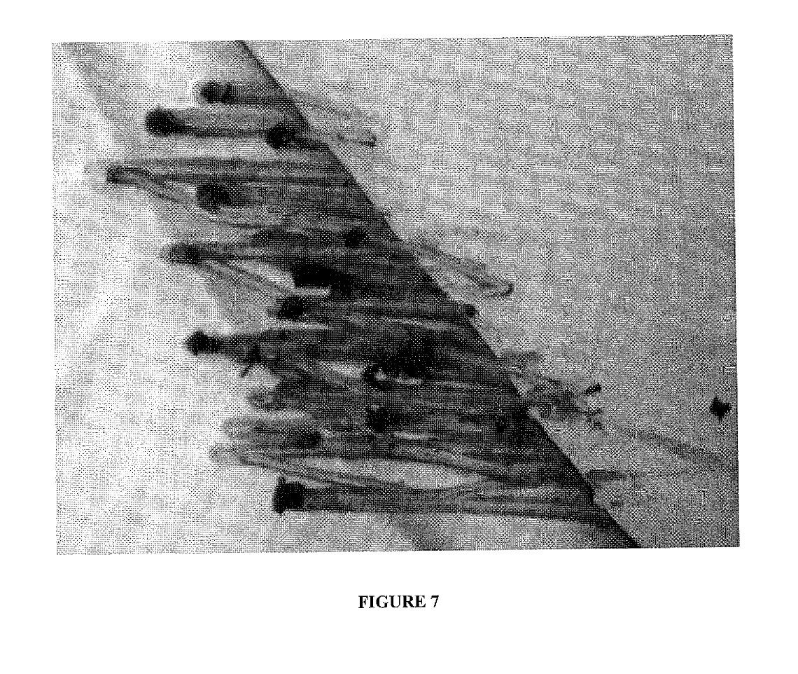

Further to the examples above, an approximate measure of the mechanical properties of a drug delivery balloon coating is accomplished by coating sample slides. FIG. 5 is a photomicrograph taken at 50× of a glass slide coating comprising zotarolimus and the liquid emulsifier Tween 20 at a weight ratio of 1/2. After dispensing 50 ul of a formulation comprising 5% zotarolimus and 10% Tween 20 in a solvent blend of acetone/methanol 47/53 (w/w), the glass slide was baked at 50° C. for one hour. The optical micrograph in FIG. 5 shows the coating after it has been scratched with a steel mandrel. The coating undergoes flow after scratching. By placing another glass slide on top of the coating and noting the force to pull them apart, the coating is also quite sticky. This behavior makes this low drug to emulsifier ratio formulation unsuitable for use as a drug delivery balloon coating. FIG. 6 is an optical micrograph at 50× of a glass slide coating made in a similar manner. This formulation was zotarolimus/PEG-PE at a 1/2 weight ratio in 100% methanol. After baking the coating at 50° C. for one hour, it was examined by a scratch test with a steel wire. Unlike the zotarolimus/Tween 20 coating at the same drug/emulsifier ratio, this coating is waxy with no trace of stickiness. These properties make it more suitable for use in a drug delivery balloon coating. FIG. 7 is another optical micrograph at 50× of a glass slide coating of a formulation of zotarolimus/vitamin E TPGS at a 2/1 weight ratio in acetone/EtOH 85/15 (w/w). Similar to the previous formulation, this coating was examined by a wire scratch test and was waxy with no evidence of stickiness or coating flow. Unlike Tween 20, both PEG-PE and vitamin E TPGS are solid emulsifiers. The fact that these emulsifiers are solids allows for a greater amount to be used without adversely affecting the coating mechanical properties. The low drug to emulsifier ratio provides for a higher amount of emulsifier to facilitate solubilization of the drug.

Emulsifier candidates can be screened by measuring the degree to which they can enhance the solubility of the therapeutic agent. One way to accomplish this is to make 5% (w/w) solutions of the emulsifiers of interest in a phosphate buffered saline solution. An excess amount of drug is added to the solution and incubated with stirring at 37° C. After centrifugation to precipitate all solids, the supernatant is assayed by HPLC for the drug concentration. Results of one such screening test are shown in Table 3.

TABLE 3
Solubility Enhancement of a Therapeutic
Agent with Select Excipients

| | Zotarolimus Solubility |
|---|---|
| Solution (5% w/w) | (ug/ml, n = 3) |
| Phosphate buffered saline | 0.53 |
| PVP C-17 | 5.6 ± 1.6 |
| Hydroxypropyl-β-cyclodextrin | 11.6 ± 3.1 |
| PEG 400 | 31.5 ± 3.5 |
| Glycerol | 43.2 ± 30.1 |
| 5% γ-Cyclodextrin | 55.3 ± 34.3 |
| Vitamin E TPGS | 512 ± 49.5 |
| Tween 20 | 732 ± 94.7 |
| 18:0 PEG2000 PE (PEG-PE)* | 1020 ± 417 |

*1,2-distearoyl-sn-glycero-3-phosphoethanolamine-N-[methoxy(polyethyleneglycol)-2000] (ammonium salt)

As illustrated in Table 3, several excipients provide for increased solubility for the cytostatic drug, zotarolimus, as compared to saline alone. The excipients Vitamin E TPGS, Tween 20, and PEG-PE demonstrate the largest increase in zotarolimus solubility. PVP provides a smaller increase in solubility. Of these, Vitamin E TPGS, PEG-PE, and PVP are solids. This allows for these excipients to be used at a lower drug:excipient ratio compared to a liquid excipient, such as Tween 20. Since glycerol and Tween 20 are a liquid, their fractions in the coating cannot be raised as high as that with the Vitamin E TPGS or PEG-PE without the coating becoming soft and tacky.

In accordance with another aspect of the disclosed subject matter, and as previously noted, a method of manufacturing a drug delivery device is provided. The drug delivery device can be, for example, a balloon with or without a stent. The method includes applying a coating including an effective amount of a therapeutic agent to an expandable member to define a coating thickness of about 0.5 μm to about 20 μm, and disposing the expandable member on a catheter. In an alternative embodiment, providing a catheter including an expandable member; and applying a coating including an effective amount of a therapeutic agent to the expandable member to define a coating thickness of about 0.5 μm to about 20 μm. As previously noted, a wide variety of catheters and balloons can be used with the method herein.

The method can further include preparing the coating, during which the preparation includes mixing a therapeutic agent, such as an effective amount of a therapeutic agent, and an excipient to form a precoating, and conditioning the precoating by a phase inversion technique to define a porous coating for application to the expandable member. Alternatively, or additionally, the method can include defining a porous coating by adding a porogen to the coating or preparing the coating by inclusion of a porogen, as described below.

In accordance with the disclosed subject matter, the coating thickness applied to a medical device or a balloon is controlled. Various techniques are available to control the coating thickness for a drug delivery balloon. For the purpose of illustration but not limitation, the coating thickness can be controlled by changing: (1) drug dose per unit of balloon surface area, (2) percent solids of drugs and excipients in the coating solution, (3) ratio of therapeutic agent to excipients in the drug formulation, (4) changing the surface area of coating per a certain dose and formulation, (5) adding porosity or void volume to the coating, or (6) particulars of the coating process such as coating method, drying rate and solvent used.

For example, the surface area can be reduced by decreasing length (L) of the balloon for a particular therapeutic agent dose and formulation. Rather than decreasing the working area of the balloon that is coated, the balloon can be coated by a series of bands wrapping around the balloon, or stripes running along the length of the balloon. Many other patterns are possible such as checkerboard or a plurality of dots. In all of these cases, the amount of drug dissolution from the balloon, rate of drug dissolution, or coating transfer to the vessel wall will be increased via an increase in coating thickness.

Other means to increase the coating thickness include: (1) increasing the therapeutic agent dose, and (2) increasing the amount of excipient for a given drug dose. While increasing the dose will render the coating more prone to fracture, during inflation there is an upper limit on the amount of therapeutic agent that can be used so as not to exceed the no observable adverse effect level (“NOAEL”), which is based on systemic drug exposure and available toxicological data for the drug.

In addition, a porous coating of the same dose would have a larger coating thickness. There are many methods to create a porous, open celled coating such as (1) incorporation of a porogen into the coating, which is subsequently leached out after the coating process (e.g., salt leaching) and (2) use of a coating which undergoes phase inversion (e.g., thermal induced phase separation). Phase inversion is a process that creates porous structures. Phase inversion either starts with a homogenous single phase solution (Sol 1), which at some point before gelation undergoes a transition into a heterogeneous solution of molecular aggregates consisting of two interdispersed liquid phases (Sol 2), or it starts with a heterogeneous solution of molecular aggregates consisting of two interdispersed liquid phases (Sol 2).

Phase inversion can be accomplished by use of a solvent and excipient blends in a drying process, a thermal process where the polymer is only soluble at an elevated temperature in the solvent, or a wet process where a dense coating is subsequently exposed to additional solvent processing. The drying process is most applicable to coatings containing a drug. A simple concept is to dissolve the drug and excipients in a solvent blend where the faster evaporating solvent is compatible solvents for the polymer and/or drug. Other examples of phase inversion techniques to produce porous surfaces include lyophilization, high pressure gas foaming, solid freeform fabrication, fiber bonding of extruded microfibers and fiber based electrospinning of micro- or nanofibers.

With reference to the balloon construction, a polymeric expandable balloon is preferred. Various polymers can be selected for the formation of the balloon, as would be known in the art. For example, the polymeric material can be a compliant, non-compliant or semi-compliant polymeric material or polymeric blend.

In one embodiment, the polymeric material is compliant such as but not limited to a polyamide/polyether block copolymer (commonly referred to as PEBA or polyether-block-amide). Preferably, the polyamide and polyether segments of the block copolymers can be linked through amide or ester linkages. The polyamide block can be selected from various aliphatic or aromatic polyamides known in the art. Preferably, the polyamide is aliphatic. Some non-limiting examples include nylon 12, nylon 11, nylon 9, nylon 6, nylon 6/12, nylon 6/11, nylon 6/9, and nylon 6/6. Preferably, the polyamide is nylon 12. The polyether block can be selected from various polyethers known in the art. Some non-limiting examples of polyether segments include poly(tetramethylene glycol), tetramethylene ether, polyethylene glycol, polypropylene glycol, poly(pentamethylene ether) and poly(hexamethylene ether). Commercially available PEBA material can also be utilized such as for example, PEBAX® materials supplied by Arkema (France). Various techniques for forming a balloon from polyamide/polyether block copolymer are known in the art. One such example is disclosed in U.S. Pat. No. 6,406,457 to Wang, the disclosure of which is incorporated herein by reference in its entirety.

In another embodiment, the balloon material is formed from polyamides. Preferably, the polyamide has substantial tensile strength, be resistant to pin-holing even after folding and unfolding, and be generally scratch resistant, such as those disclosed in U.S. Pat. No. 6,500,148 to Pinchuk, the disclosure of which is incorporated herein by reference in its entirety. Some non-limiting examples of polyamide materials suitable for the balloon include nylon 12, nylon 11, nylon 9, nylon 69 and nylon 66. Preferably, the polyamide is nylon 12. In yet another embodiment, the balloon is composed of several different layers, each a one a different polyamide or polyamide/polyether block copolymer. Other suitable materials for constructing non-compliant balloons are polyesters such as poly(ethylene terephthalate) (PET), Hytrel thermoplastic polyester, and polyethylene.

In another embodiment, the balloon can be formed a polyurethane material, such as TECOTHANE® (Thermedics). TECOTHANE® is a thermoplastic, aromatic, polyether polyurethane synthesized from methylene disocyanate (MDI), polytetramethylene ether glycol (PTMEG) and 1,4 butanediol chain extender. TECOTHANE® grade 1065D is presently preferred, and has a Shore durometer of 65D, an elongation at break of about 300%, and a high tensile strength at yield of about 10,000 psi. However, other suitable grades can be used, including TECOTHANE® 1075D, having a Shore D of 75. Other suitable compliant polymeric materials include ENGAGE® (DuPont Dow Elastomers (an ethylene alpha-olefin polymer) and EXACT® (Exxon Chemical), both of which are thermoplastic polymers. Other suitable compliant materials include, but are not limited to, elastomeric silicones, latexes, and urethanes. The compliant material can be cross linked or uncrosslinked, depending upon the balloon material and characteristics required for a particular application. The presently preferred polyurethane balloon materials are not crosslinked. However, other suitable materials, such as the polyolefinic polymers ENGAGE® and EXACT®, are preferably crosslinked. By crosslinking the balloon compliant material, the final inflated balloon size can be controlled. Conventional crosslinking techniques can be used including thermal treatment and E-beam exposure. After crosslinking, initial pressurization, expansion, and preshrinking, the balloon will thereafter expand in a controlled manner to a reproducible diameter in response to a given inflation pressure, and thereby avoid overexpanding the stent (when used in a stent delivery system) to an undesirably large diameter. In one embodiment, the balloon is formed from a low tensile set polymer such as a silicone-polyurethane copolymer. Preferably, the silicone-polyurethane is an ether urethane and more specifically an aliphatic ether urethane such as PURSIL AL 575A and PURSIL AL10, (Polymer Technology Group), and ELAST-EON 3-70A, (Elastomedics), which are silicone polyether urethane copolymers, and more specifically, aliphatic ether urethane cosiloxanes. In an alternative embodiment, the low tensile set polymer is a diene polymer. A variety of suitable diene polymers can be used such as but not limited to an isoprene such as an AB and ABA poly(styrene-block-isoprene), a neoprene, an AB and ABA poly(styrene-block-butadiene) such as styrene butadiene styrene (SBS) and styrene butadiene rubber (SBR), and 1,4-polybutadiene. Preferably, the diene polymer is an isoprene including isoprene copolymers and isoprene block copolymers such as poly(styrene-block-isoprene). A presently preferred isoprene is a styrene-isoprene-styrene block copolymer, such as Kraton 1161K available from Kraton, Inc. However, a variety of suitable isoprenes can be used including HT 200 available from Apex Medical, Kraton R 310 available from Kraton, and isoprene (i.e., 2-methyl-1,3-butadiene) available from Dupont Elastomers. Neoprene grades useful in the disclosed subject matter include HT 501 available from Apex Medical, and neoprene (i.e., polychloroprene) available from Dupont Elastomers, including Neoprene G, W, T and A types available from Dupont Elastomers.

The balloon can be composed of a single polymeric layer, or alternatively, can be a multilayered balloon, such as those described in U.S. Pat. No. 5,478,320 to Ishida, U.S. Pat. No. 5,879,369 to Trotta, or U.S. Pat. No. 6,620,127 to Lee, the disclosures of which are incorporated herein by reference in their entirety.

In a preferred embodiment, the outer surface of the balloon is textured. In this regard, the balloon surface can include a roughened surface, voids, spines, or microcapsules or a combination thereof, as will be described below.

In another embodiment of the disclosed subject matter, the balloon is formed of a porous elastomeric material having at least one void formed in the wall of the balloon surface. The entire cross section of the balloon can contain a plurality of voids. Alternatively, the plurality of void can be distributed along select lengths of the balloon outer surface. For example, and not by way of limitation, the plurality of voids can be distributed only along the working section of the balloon. The voids define an open space within the outer surface of the balloon. Preferably, the therapeutic agent is dispersed within the space defined by the plurality of voids across the cross section of the balloon outer surface.

In operation, the therapeutic agent is released or is expelled from the pores upon inflation of the balloon. In this regard, the durometer of the polymeric material of the balloon surface and in particular the depression of the void is sufficiently flexible to allow for expulsion of the therapeutic agent and/or coating contained within the plurality of voids upon inflation of the balloon. The expelled coating with therapeutic agent is released into the vessel lumen or into the tissue surrounding and contacting the inflated balloon.

In another embodiment, the balloon includes protrusions configured to contact or penetrate the arterial wall of a vessel upon inflation of the balloon. A coating containing therapeutic agent is disposed on the protrusions and when inflated the coating and/or therapeutic agent coats the tissue of the arterial wall. Alternatively, the balloon can include two concentric balloons in a nesting configuration. The coating with therapeutic agent is disposed between the two concentric balloons. Thus, the space between the two concentric balloons, one being an interior balloon and the other being an exterior balloon, acts as a reservoir. In this regard, the protrusions can include apertures for expulsion of the coating and/or therapeutic agent upon inflation of the interior and exterior concentric balloons. For example, as described in U.S. Pat. No. 6,991,617 to Hektner, the disclosure of which is incorporated herein by reference in its entirety. In another embodiment, the balloon can include longitudinal protrusions configured to form ridges on the balloon surface. As described in U.S. Pat. No. 7,273,417 to Wang, the disclosure of which is incorporated herein by reference in its entirety, the ridges can be formed of filaments spaced equidistantly apart around the circumference of the balloon. However, a larger or smaller number of ridges can alternatively be used. The longitudinal ridges can be fully or partially enveloped by the polymeric material of the balloon.

In yet another embodiment, the balloon can include microcapsules on its outer surface. In this regard, the microcapsules are configured to encompass the coating and/or therapeutic agent. Upon inflation of the balloon the microcapsules located on the surface of the balloon contact the tissue of the arterial wall. Alternatively, the microcapsules can be formed in the wall of the balloon surface. The coating and/or therapeutic agent can be released from the microcapsules by fracturing of the microcapsules and/or diffusion from the microcapsule into the arterial wall. The microcapsules can be fabricated in accordance with the methods disclosed in U.S. Pat. No. 5,1023,402 to Dror or U.S. Pat. No. 6,129,705 to Grantz and the patents referenced therein, each of which is incorporated herein by reference in their entirety.

In accordance with another aspect, if desired, a protective sheath can be utilized to protect the coating from being rubbed off of the balloon during the movement of the coated balloon through the body lumen. The sheath is preferably made from an elastic and resilient material that conforms to the shape of the balloon and in particular is capable of expanding upon inflation of the balloon. The sheath preferably includes apertures along a length thereof. In operation, the inflation of the balloon causes the apertures of the sheath to widen for release of the coating and/or therapeutic agent to the tissue of the arterial wall. Preferably, the sheath has a thickness less than about 10 mils. However, other thicknesses can be used.

In another embodiment, the sheath has at least one longitudinal line of weakness allowing the sheath to rupture upon inflation of the balloon and the release of the coating and/or therapeutic agent onto the tissue of the arterial wall of the vessel. Preferably, the sheath is formed from polymeric material known to be suitable for use in balloon catheters. Preferably, the sheath material is an elastomeric material that will also spring back when it splits to expose more of the body lumen to the coating. The line of weakness could be provided by various. techniques known in the art. However, one non-limiting example includes perforating the sheath material. In operation, the sheath is placed over the coated balloon while in the deflated state. When the coated balloon inflated, the sheath is expanded to the extent that it exceeds its elastic limit at the line of weakness and bursts to expose and therefore release the coating and/or therapeutic agent to the tissue of the arterial wall or vessel lumen. For example, see U.S. Pat. No. 5,370,614 to Amundson, the disclosure of which is incorporated by reference in its entirety.

The disclosed subject matter is not to be limited in scope by the specific embodiments described herein. Indeed, various modifications of the disclosed subject matter in addition to those described herein will become apparent to those skilled in the art from the foregoing description and the accompanying figures. Such modifications are intended to fall within the scope of the appended claims.

Patents, patent applications, publications, procedures, and the like are cited throughout this application, the disclosures of which are incorporated herein by reference in their entireties.

What is claimed is:

1. A balloon for delivering a therapeutic agent to a vessel wall, the balloon comprising:
a body having a working length disposed between a distal end and a proximal end thereof; and
a coating applied to at least a length of the body, wherein the coating includes a hydrophobic therapeutic agent having a water solubility less than about 15.0 μg/ml and an emulsifier, wherein the emulsifier is a solid at ambient temperature.

2. The balloon of claim 1, wherein the coating has a weight ratio of hydrophobic therapeutic agent to emulsifier of about 3:1.

3. The balloon of claim 1, wherein the coating has a weight ratio of hydrophobic therapeutic agent to emulsifier of less than 3:1.

4. The balloon of claim 1, wherein the hydrophobic therapeutic agent is a cytostatic drug.

5. The balloon of claim 1, wherein the hydrophobic therapeutic agent is selected from the group consisting of zotarolimus, everolimus, sirolimus or derivatives thereof, biolimus, novolimus, myolimus, temsirolimus, deforolimus, paclitaxel or derivatives thereof, docetaxel, protaxel, and taxanes.

6. The balloon of claim 1, wherein the emulsifier is selected from the group including Tween 60, Vitamin E TPGS, Pluronic F68, Pluronic F127, Poloxamer 407, poly(vinyl pyrrolidone), Ascorbyl palmitate lecithin, egg yolk phospholipid, phosphatidylcholine, polyethylene glycol-phosphatidyl ethanolamine conjugate or a combination thereof.

7. The balloon of claim 1, wherein the emulsifier is a PEG-Phospholipid conjugate.

8. The balloon of claim 1, wherein the coating further includes a plasticizer.

9. The balloon of claim 8, wherein the plasticizer is a liquid at ambient temperature.

10. The balloon of claim 9, wherein the plasticizer is a polysorbate.

11. The balloon of claim 10, wherein the polysorbate is Tween 20 or Tween 80.

12. The balloon of claim 9, wherein the plasticizer is an oil.

13. The balloon of claim 12, wherein the oil is selected from the group consisting of soybean oil, peanut oil, safflower oil, poppyseed oil, vegetable oil, cottonseed oil, castor oil, and almond oil.

14. The balloon of claim 9, wherein the plasticizer is selected from the group consisting of benzyl alcohol, DMSO, NMP, glycerol, propylene glycol, Cremophor EL, Vitamin E, tocopherol, ethyl lactate, and liquid PEG.

15. The balloon of claim 9, wherein the plasticizer is no more than about 20% by weight of the total solids of the coating.

16. The balloon of claim 1, wherein the hydrophobic therapeutic agent is paclitaxel and the emulsifier is Tween 60.

17. The balloon of claim 16, wherein the coating further comprises a plasticizer, and further wherein the plasticizer is Cremophor EL.

18. The balloon of claim 1, wherein the therapeutic agent is zotarolimus, and the emulsifier is 18:0 PEG2000 PE.

19. The balloon of claim 1, wherein the therapeutic agent is everolimus, and the emulsifier is Vitamin ETPGS.

20. The balloon of claim 1, wherein the therapeutic agent is zotarolimus, and the emulsifier is polyethylene glycol-phosphatidyl ethanolamine conjugate.

21. The balloon catheter of claim 1, wherein a stent is disposed on the balloon.

22. A drug coated balloon comprising:
a body having a working length disposed between a distal end and a proximal end thereof; and
a coating applied to at least a length of the body, wherein the coating includes a cytostatic therapeutic agent having a water solubility less than about 15.0 μg/ml and an emulsifier, wherein the emulsifier is a solid at ambient temperatures, and further wherein the ratio of the cytostatic therapeutic agent to emulsifier is less than 3:1.

23. A method of manufacturing a drug delivery balloon comprising:
providing a catheter including an expandable balloon member; and
applying a solution comprising a hydrophobic therapeutic agent having a water solubility less than 15.0 μg/ml and an emulsifier having properties of a solid at ambient temperature to the expandable balloon member; and
heating the balloon to remove solvent.

24. The method of claim 23, wherein the balloon is inflated to a low pressure of about 0.2 to about 9 atm.

25. The method of claim 23, wherein heating includes baking the balloon at a temperature greater than about 50° C.

26. The method of claim 23, wherein the balloon is rotated and translated while applying the solution.

27. The method of claim 23, wherein heating occurs for about 30 to about 60 minutes.

28. The method of claim 23, wherein the solution further contains a plasticizer, wherein the plasticizer is a liquid at ambient temperature.

29. The method of claim 23, further comprising crimping a stent on the expandable balloon member.
